(19) United States
(12) Patent Application Publication
(10) Pub. No.: US 2010/0015025 A1
(43) Pub. Date: Jan. 21, 2010

(54) PROCESS FOR PREPARING HYDROXYLAPATITE

(75) Inventors: Gavin Walker, Belfast (GB); Fraser Buchanan, Belfast (GB); Pamela Walsh, Belfast (GB); Christine Maggs, Belfast (GB); Matt Dring, Belfast (GB)

Correspondence Address: PEPPER HAMILTON LLP, ONE MELLON CENTER, 50TH FLOOR, 500 GRANT STREET, PITTSBURGH, PA, 15219, US

(73) Assignee: THE QUEEN'S UNIVERSITY OF BELFAST, Belfast (GB)

(21) Appl. No.: 12/300,848

(22) PCT Filed: May 17, 2007
(86) PCT No.: PCT/GB2007/001844

§ 371 Date: Jul. 10, 2009

(30) Foreign Application Priority Data
May 18, 2006 (GB) 0609815.6

(51) Int. Cl. C12P 3/00; C12N 1/12; C01B 25/32

(52) U.S. Cl. 423/308; 435/168; 435/257.1

(57) ABSTRACT

A process for preparing hydroxylapatite from a calcium carbonate-containing algae comprising the steps of: (a) converting at least some of the calcium carbonate in the algae to calcium oxide without changing the porosity of the algae; and (b) reacting the so-formed material of step (a) with phosphate ions in water.

The present invention relates to a process for preparing hydroxylapatite, an intermediate material in said process, and the hydroxylapatite material formed.

Tissue engineering has emerged as an alternative approach to circumvent the existent limitations in the current therapies for organ failure or replacement, which are mainly related with the difficulty of obtaining tissues or organs for transplantation. Conventional material technology has resulted in clear improvements in the field of regeneration/substitution medicine. However, despite the good results with the current methodologies, due to their severity, most of these injuries are still unrecoverable, creating a major healthcare problem world wide.

Bone tissue engineering provides a viable approach to the replacement of damaged or diseased tissue in the form of a three-dimensional tissue specific cell scaffolds.

In scaffold fabrication, biomaterials play a pivotal role as they instigate the growth of cell culture. Ideally, they need to stimulate an osteoblastic response via an interaction with specific adhesion and growth receptors by targeting cells from the host tissue. To achieve this, the biomaterial needs a viable means to support angiogenesis that will distribute nutrients and diffuse gases at the site of repair. Natural hydroxylapatite (HA) from marine coral derivates hosts natural architecture of interconnecting pores which may serve as an osteoconductive structure, promoting cell adhesion and proliferation.

However, biomaterials used in scaffold fabrication also need a resorption rate that is synchronised with the ossification of new bone, a property, which crystalline HA lacks. Beta tri-calcium phosphate (βTCP) offers an alternative to HA, but with a significantly faster resorption. By creating a biphasic HA/TCP structure, a material with a specifically tailored resorption rate, to mimic that of nature bone, may be created.

US 20002114755 discloses a method of producing a hydroxylapatite material containing tricalcium phosphate from a hard algae tissue by pyrolising the algae for 24 hours at 700° C., and then reacting the so-formed material at a temperature of above 150° C., usually 230-250° C., for at least another 24 hours, at an increased pressure in an autoclave.

It is an object of the present invention to provide hydroxylapatite using a more efficient process.

Thus, according to one aspect of the present invention, there is provided a process for preparing hydroxylapatite from a calcium carbonate-containing algae comprising the steps of:

(a) converting at least some of the calcium carbonate in the algae to calcium oxide without changing the porosity of the algae; and
(b) reacting the so-formed material of step (a) with phosphate ions in water.

An advantage of the present invention is to provide a process which is significantly simplified from prior methods of providing hydroxylapatite from previous sources.

The calcium carbonate-containing algae may be any suitable algae known to contain a significant portion of calcium carbonate Many calcified species of algae are known, and include Amphiroa ephedraea and other members of the algae family Corallinaceae, as well as some siphonous green algae in the green family Codiaceae. Other coralline species of algae are known. One particularly suitable material is the geniculate (jointed) species Corallina officinalis. Other non-jointed coralline species of algae are known, including encrusting forms and free-living rhodolith (maerl) forms.

The algae is intended to have a porosity which is wholly or substantially (for example >75%, >80%, >85%, 90%, 95% or 97% or >98%) the same or similar to the porosity of human bone. In general, this can be defined as having micro-millimetre pore sizes, such as being in the range 10-1000 micron.

In step (a), the calcium carbonate-containing algae can be converted by heating.

Heating calcium carbonate-based algae, sometimes also termed ‘charring’, can be carried out using a suitable temperature regime. Preferably, the heating temperature is between 600-800° C., preferably 630-720° C., more preferably 650-700° C., such temperature being able to provide the right conditions for changing a proportion of the calcium carbonate to calcium oxide. Also preferably, the heating is carried out at ambient pressure.

The conversion of calcium carbonate in the calcium carbonate-containing algae to calcium oxide is preferably at least 5-10 wt %, more preferably 15-25 wt %, 18-22 wt %, 19-21 wt % and even more preferably approximately 20 wt %.

In step (a), it is preferable to at least partly remove carbon in the calcium carbonate-containing seaweed. More preferably, it is intended to remove >95 wt % of carbon, more preferably >99 wt %.

The so-formed intermediate material formed by step (a) still possesses the micro-porous structure of the original algae material. However, the thermal treatment has decomposed some of the calcium carbonate to calcium oxide compared with the original algae which contains 100% calcium carbonate

In step (b), the phosphate ions can be provided in any suitable form, generally in solution. Many soluble phosphate compounds are known. In step (b), heat is preferably also used, such as can heat a phosphate solution to approximately 100° C., for example in the range 80-120° C.

The phosphate ions used in step (b) can be provided as an aqueous phosphate solution, preferably of diammonium hydrogen phosphate [(NH₄)₂HPO₄] and magnesium nitrate [Mg(NO₃)₂.6H₂O]. The pH of the solution may require regulation (before phosphate solution addition) within the range of 9.0-9.5, such as by using ammonium hydroxide [NH₄OH].

After the reaction is completed, the pH of solution is preferably measured to ensure it remained within this range.

The present invention extends to a hydroxylapatite material whenever prepared by a process as hereinbefore described.

The present invention also extends to a treated calcium-carbonate-containing algae material wherein >95 wt %, preferably >99 wt % of carbon has been removed, and 5-40 wt %, more preferably 5-15 wt % of the calcium carbonate has been formed to calcium oxide.

The present invention also extends to an intermediate or so-formed material of step (a) as hereinbefore defined in the process as hereinbefore defined.

The present invention also extends to use of the intermediate material of step (a) as hereinbefore defined in the process for preparing a hydroxylapatite material as hereinbefore defined.

The present invention also extends to use of a hydroxylapatite material as herein before defined in tissue engineering, and particularly for use in tissue engineered scaffold fabrication.

An embodiment of the present invention will now be described by way of example only. Reference is made to the accompanying figures and drawings in which:

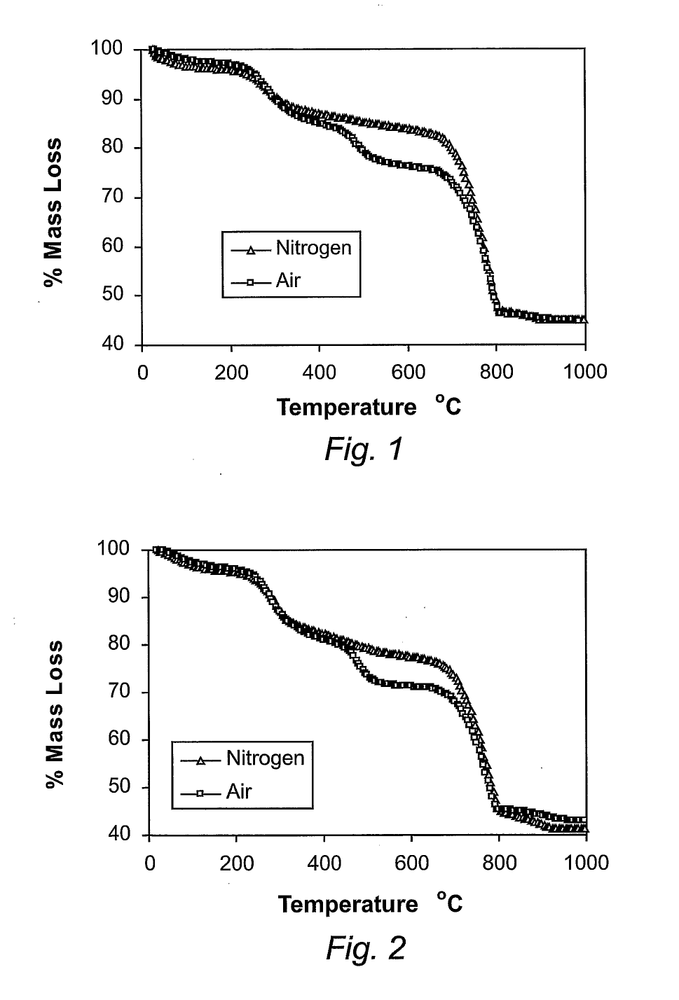

FIG. 1 shows a non-isothermal analysis of Corallina officinalis under N₂ and air atmospheres;

FIG. 2 shows a non-isothermal analysis of Amphiroa ephedraea under N₂ and air atmospheres;

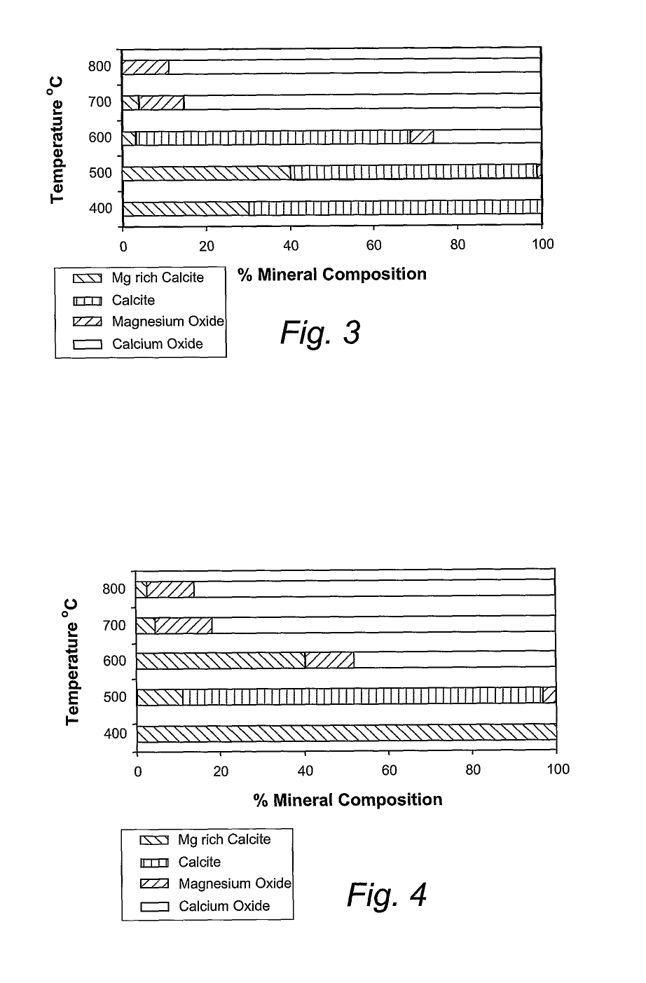

FIG. 3 demonstrates the chemical composition (normalised) of Corallina officinalis after pyrolysis for 12 hours at different temperatures using a ramp rate of 0.5° C./min;

FIG. 4 demonstrates the chemical composition (normalised) of Amphiroa ephedraea after pyrolysis for 12 hours at different temperatures using a ramp rate of 0.5° C./min;

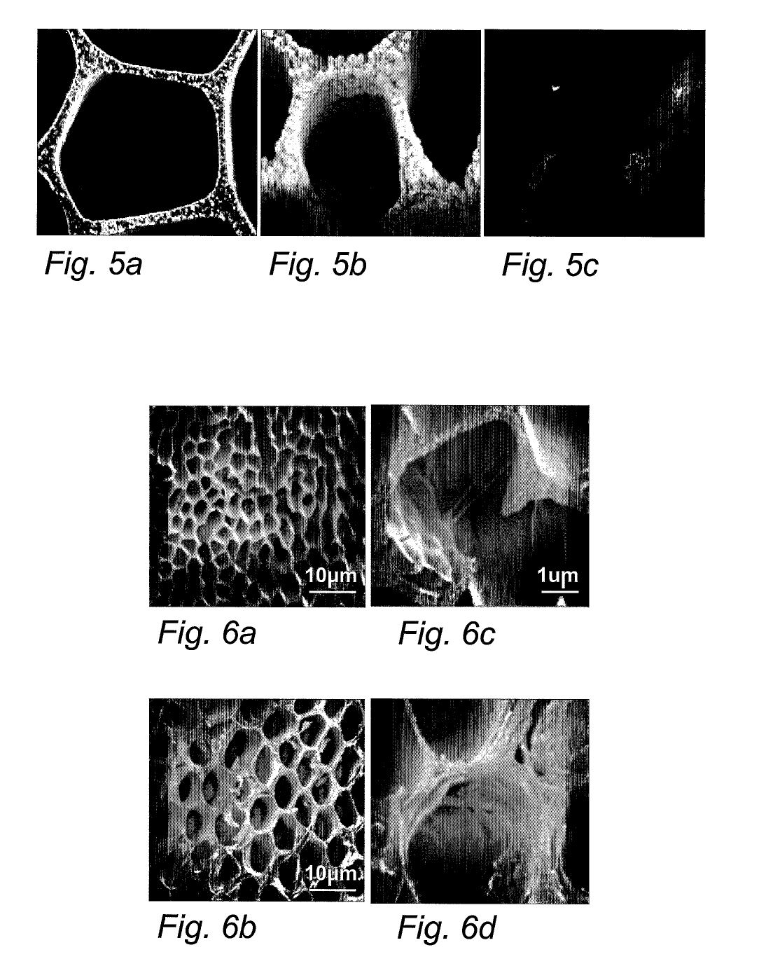

FIG. 5 (a-c) are micrographs of the internal cross-sections of Amphiroa ephedraea after treating at (a) 600° C. (b) 700° C. and (c) 800° C.;

FIG. 6 (a-d) are micrographs of the internal morphology of Corallina officinalis (a) raw algae perpendicular to pore orientation (b) after heat treatment at 650° C.;

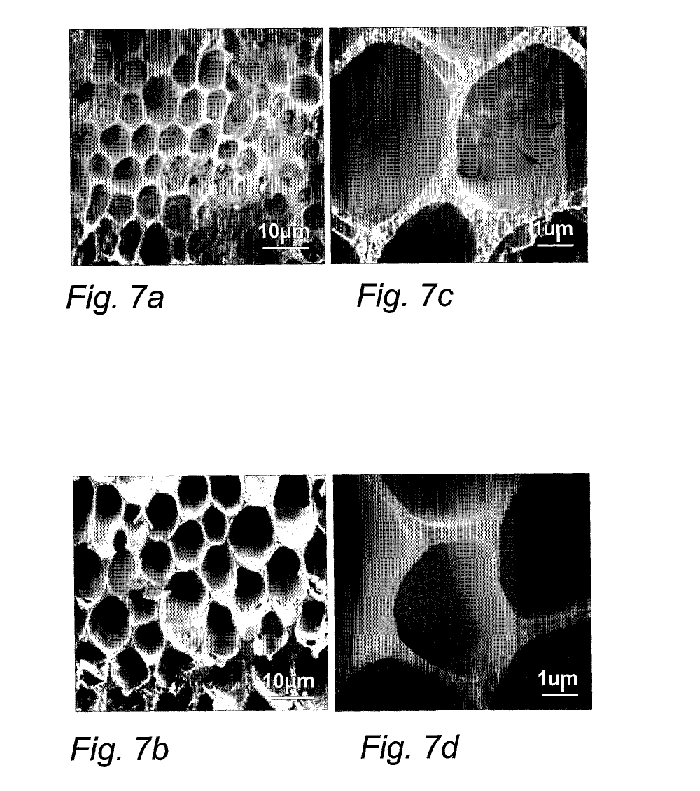

FIG. 7 shows a micrograph of internal morphology of Amphiroa ephedraea (a) raw algae perpendicular to pore orientation (b) after heat treatment at 650° C.; and

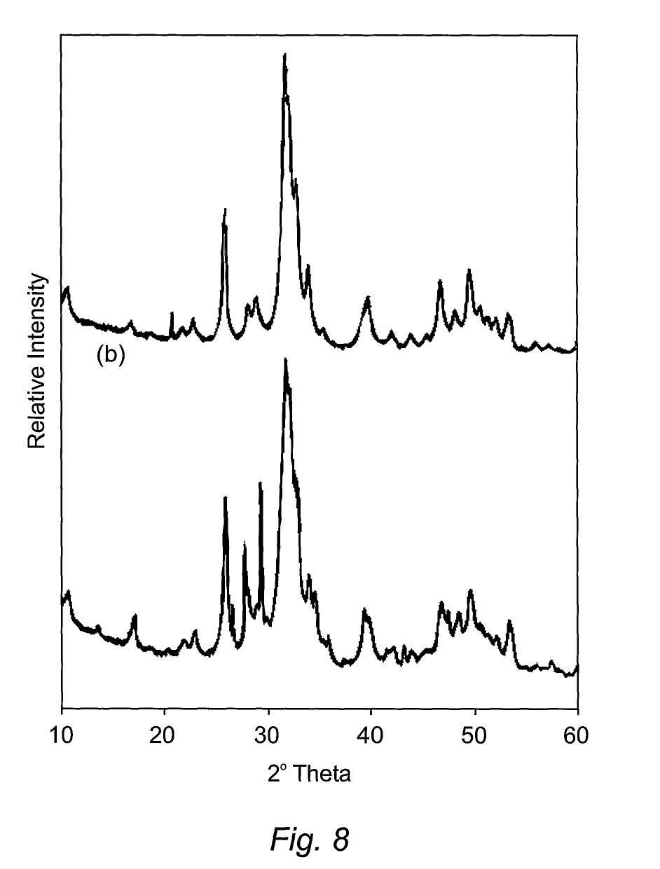

FIG. 8 demonstrates XRD traces of hydroxylapatite derived from (a) Amphiroa ephedraea (b) Corallina officinalis.

### EXAMPLE 1

Corallina officinalis and Amphiroa ephedraea were collected from County Donegal, Ireland. They were separately converted to hydroxylapatite by way of a two-step hydrothermal process. The first step involved a heat treatment, pyrolising the Corallina officinalis and Amphiroa ephedraea in air at 650° C. for a fixed period 12 hours to remove organic content, especially the removal of all carbon to >99 wt %. The resulting material, predominantly comprising calcium oxide, was then synthesised at atmospheric pressure and ambient temperature (100° C.). The reaction was carried out in a 1-litre reaction flask, and continuously mixed at a speed of 100 rpm in aqueous phosphate solution preferably of diammonium hydrogen phosphate [(NH₄)₂HPO₄].

Thermogravimetric analysis (TGA) was used to determine the mass loss of the Corallina officinalis and Amphiroa ephedraea as a function of temperature and time, to establish the optimum processing parameters for pyrolysis. A non-reactive (N₂) and reactive (air) atmosphere was used to distinguish between (1) the mass loss due to organic decomposition and (2) mass loss due to inorganic phase transformation. FIGS. 1 and 2 show the thermal decomposition of Corallina officinalis and Amphiroa ephedraea respectively. The first stage of decomposition occurs at <200° C. in both species, which can be attributed to the dissociation of water from the alga material. In the next stage of decomposition, between 220° C. and 650° C. two different reactions occur. Under N₂ the alga has a gradual slope as it tends asymptotically to the maximum decomposition, whereby the reaction described in equation 1 below, takes place. Under air a steeper slope occurs as a result of organic burn off. The difference in mass loss between gradients of the slopes at 650° C. was 27% (Corallina) and 21% (amphiroa) suggesting the organic content of the alga to be in that region. Between 600 to 800° C., the carbon (C) from calcium carbonate (CaCO₃) is oxidised and off-gassed as carbon dioxide (CO₂) leaving calcium oxide (CaO) resulting in the reaction described in equation 2.

Mg,Ca(CO₃)₂→CaCO₃+MgO+CO₂ (1)

CaCO₃→CaO+CO₂ (2)

The X-ray diffraction (XDR) results in FIGS. 3 and 4 show the chemical composition at different stages of thermal decomposition. The optimum processing conditions were described to be in the range of 600 to 800° C. according to TGA. The XRD results indicated that optimum level of calcium oxide required for the conversion is achieved between 600 and 700° C.

The effect of the heat treatment on the structure of the two alga was determined using SEM. The results in FIG. 5 for Amphiroa ephedraea suggest the onset of structural decomposition occurred ≧700° C. This is also true for Corallina officinalis.

The micrograph images in FIGS. 6 and 7 compare a cross-section of the two alga before and after heat treatment. In FIGS. 6a and 7a the organic matter clinging to the cell walls is evident. After heating to 650° C. this bubbly substance is no longer present and the structure has remained intact.

After synthesis XRD (FIG. 8) confirms the presence of hydroxylapatite with its characteristic three peaks residing in the region of 320 (2⁰Theta). The resultant material is described in formula 1 for the Amphiroa ephedraea species and in formula 2 for Corallina officinalis.

Ca₅(PO₄)₆(OH)₂ (1)

Ca_{9.74}(PO₄)₆(OH)_{2.08} (2)

In conclusion, the present invention provides hydroxylapatite using a more efficient and simplified process than is currently available. The process includes a two step hydrothermal method for converting calcium carbonate algae to hydroxylapatite whilst retaining the crucial micro-porous structure of the original algae akin to that of bone. The process also provides a biomaterial which possesses a suitable resorption rate to mimic that of bone and thus provides an excellent scaffold which can be used in tissue engineering, and particularly for use in tissue engineered scaffold fabrication.

1. A process for preparing hydroxylapatite from a calcium carbonate-containing algae comprising: converting at least some of the calcium carbonate in the algae to calcium oxide without changing the porosity of the algae to form an intermediate material; and reacting the intermediate material with phosphate ions in solution.

2. The process of claim 1 wherein the calcium carbonate-containing algae is selected from the group consisting of: members of the algae family Corallinaceae, siphonous green algae in the green family Codiaceae, coralline species of algae, jointed coralline species of algae, non-jointed coralline species of algae, encrusting forms of algae free-living rhodolith (maerl) forms of algae, and any combination thereof.

3. The process of claim 1 wherein the algae is Corallina officinalis.

4. The process of claim 1 wherein the algae is Amiphiroa ephedraea.

5. The process of claim 1 wherein the algae has a porosity is in the range 10-1000 microns.

6. The process of claim 1 wherein the calcium carbonate-containing algae is converted by heating.

7. The process of claim 6 wherein the heating is carried out between 650-700° C.

8. The process claim 6 wherein the heating is carried out at ambient pressure.

9. The process of claim 1 wherein the conversion of calcium carbonate in the calcium carbonate-containing algae to calcium oxide is at least 5%-10% by weight.

10. The process of claim 1 wherein the conversion of calcium carbonate in the calcium carbonate-containing algae to calcium oxide is in a range selected from the group consisting of 15%-25% by weight, 18%-22% by weight, 19%-21% by weight, more preferably and 20% by weight.

11. The process of claim 1 further comprising removing carbon in the calcium carbonate-containing algae.

12. The process of claim 11 wherein >95% by weight of carbon in the calcium carbonate-containing algae is removed.

13. The process of claim 1 wherein the intermediate material possesses the same micro-porous structure of the algae

14. The process of claim 1 wherein the phosphate ions are provided in an aqueous solution.

15. The process of claim 1 wherein reacting the intermediate material comprises heating.

16. The process as of claim 15 wherein heating comprises bringing a phosphate solution to approximately 100° C.

17. The process of claim 14 wherein the phosphate ions are provided as an aqueous phosphate solution of diammonium hydrogen phosphate [(NH₄)₂HPO₄].

18. The process of claim 17 wherein the phosphate solution also comprises magnesium ions.

19. The process of claim 14 wherein the pH of the solution is within a range of 9.0-9.5.

20. A hydroxylapatite material obtained by the process of claim 1.

21. An intermediate material obtained by the process of claim 1.

22. A treated calcium-carbonate-containing algae material wherein >95% by weight of carbon has been removed, and wherein >5%-40% by weight of the calcium carbonate has been converted to calcium oxide.

23. Use of a hydroxylapatite material obtained by the process of claim 1 for use in tissue engineering or tissue engineered scaffold fabrication.
